(19) United States
(12) Patent Application Publication
(10) Pub. No.: US 2018/0023095 A1
(43) Pub. Date: Jan. 25, 2018

(54) DATA LOGGING DEVICE

(71) Applicant: ARM Ltd, Cambridge (GB)

(72) Inventors: Jonathan Curtis Beard, Austin, TX (US); Gary Dale Carpenter, Austin, TX (US)

(21) Appl. No.: 15/218,838

(22) Filed: Jul. 25, 2016

(51) Int. Cl. C12N 15/90; G01N 33/53

(52) U.S. Cl. CPC C12N 15/902; G01N 33/5308

(57) ABSTRACT

A device and a method to sense changes in the environment and log the sensed changes.

### TECHNICAL FIELD

The present invention generally relate to methods, apparatus and systems for data logging, and in particular to a device for sensing changes in the environment and logging the sensed changes.

### BACKGROUND ART

Environmental sensors typically sense and collect data from the environment in which they are placed, and may be able to continuously operate over a wide range of time periods. Some sensors may operate for a period of months, while others may function for years or decades. Sensors which operate for a long period of time may be useful, because they can be left to collect data that can be analyzed many years later. For example, sensors may be used to monitor a slowly-changing environmental condition, such as a gradual change in global temperature or a gradual change in sea water salinity. However, it can be difficult to keep the sensors powered for such lengths of time, and/or recovering data from the sensors may be difficult (e.g. if the data is being stored in a storage medium or format that later becomes redundant).

### SUMMARY OF THE INVENTION

Accordingly, the present applicant has recognized the need for an improved sensor and data logging mechanism.

According to one embodiment of the present invention there is provided a data logging device comprising: at least one sensor to detect an environmental event; a biological data store; and at least one write mechanism to, responsive to detection of the environmental event, write data into the biological data store.

According to a second embodiment of the present invention, there is provided a system comprising: a data logging device as described herein, and a read mechanism to read data stored in the biological data store.

According to a third embodiment of the present invention, there is provided a method of logging data, comprising: detecting, using at least one sensor, an environmental event; writing, responsive to the detecting, data into a biological data store.

### BRIEF DESCRIPTION OF THE DRAWINGS

Embodiments of the invention are diagrammatically illustrated, by way of example, in the accompanying drawings, in which:

FIG. 1 is a block diagram of a system for sensing, logging and reading data;

FIG. 2 illustrates example steps to log (i.e. write) data or to add a timestamp into a biological data store;

FIG. 3 illustrates an example clocking mechanism comprising a two gene oscillator (graph), and a schematic of how data and timestamps are added to a biological data store;

FIG. 4a illustrates an example clocking mechanism comprising a two gene oscillator and FIG. 4b illustrates an example clocking mechanism comprising a three gene oscillator;

FIG. 5 is a flow diagram of example steps to write data in response to sensing an event;

FIG. 6 is a schematic diagram of how a three gene oscillator is used to add timestamps to a biological data store; and

FIG. 7 is a schematic diagram of how a data logging device may be used in an Internet of Things system.

### DETAILED DESCRIPTION

Broadly speaking, embodiments of the present invention provide a device and a method to sense changes in the environment and log the sensed changes. In particular, the device comprises biological components and uses biological processes to sense and log data. For example, the device comprises a biological data store which may be used to write and store data for long periods of time (e.g. years, decades or longer), and which may be read using biological techniques. The device may be able to autonomously sense changes and log the sensed changes. The device may be self-assembling and self-maintaining, and may be able to function in an environment for long periods without user action. In comparison to traditional sensors, which are often battery or mains-powered, the device described herein may be reliably deployed in an environment for long periods of time since the device does not require a battery or mains-connection. In embodiments, the device may be self-replicating (i.e. may be able to multiply/reproduce).

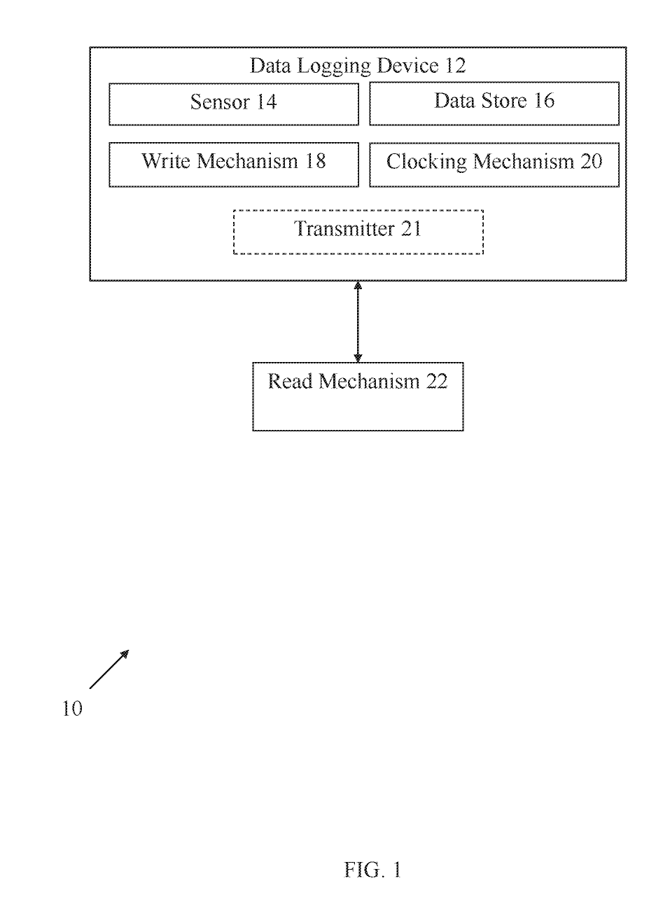

FIG. 1 is a block diagram of a system 10 for sensing, logging and reading data. The system 10 comprises a data logging device 12. In embodiments, the data logging device may be one or more of: autonomous, self-assembling, self-maintaining, and self-replicating. In embodiments, the data logging device 12 may be a biological cell (e.g. a living, naturally-occurring cell), which may be engineered to provide particular functions. In embodiments, the data logging device 12 may be a synthetic cell (also referred to herein as an artificial cell or a minimal cell) which is engineered to provide particular functions of a biological cell. In embodiments where the data logging device 12 is a natural or synthetic cell, the device 12 may be able to self-replicate, e.g. via a cell division process.

The data logging device 12 may sense one or more environmental events that occur in the environment in which the device 12 is located. The data logging device 12 comprises at least one sensor 14 to detect an environmental event. For example, the sensor 14 may detect any one of (or changes in) pH, light, wavelength, electromagnetic radiation, and concentration of a molecule or ion, though it will be understood that this is a non-exhaustive list. Examples of how the data logging device may be used are described below.

In embodiments, the sensor 14 may be (or may comprise) a receptor, i.e. a protein/molecule that is able to receive signals external to the cell. In this case, the receptor may be able to receive signals from outside the data logging device 12. For example, the receptor may comprise one of the G protein-coupled receptor (GPCR) family of receptors. GPCR receptors are capable of detecting/sensing light-sensitive compounds, odors, pheromones, hormones, and particular molecules. The GPCR receptor may be provided anywhere within the data logging device 12. In embodiments, the sensor 14 may comprise a cell surface receptor, which may be provided at, on, or near a surface of the data logging device 12. A cell surface receptor is a protein that is able to receive signals external to the cell and is built into the cell membrane. In embodiments, the sensor 14 may comprise a natural receptor or an artificial/synthetic receptor. Synthetic receptors may be designed to detect specific biomolecules, such as inorganic cations, organic and inorganic anions, carbohydrates, amino acids and peptides, proteins, lipids, etc.

The data logging device 12 may be engineered to sense one or more external, environmental events, and therefore, may comprise a sensor 14 for each event to be sensed. For example, the data logging device 12 may be used to sense changes in pH and salinity, and therefore may comprise one sensor 14 for pH and another sensor 14 for salinity. In each case, the sensor 14 may be provided by a receptor, cell surface receptor, a proton pump (such as a light-driven proton pump), or any other suitable biological element capable of sensing environmental events. In a particular non-limiting example, the sensor 14 may be a light-driven proton pump (e.g. bacteriorhodopisin) which senses particular wavelengths of light, and initiates proton pumping in response to the sensing. The effect of the sensor 14 sensing/detecting a particular environmental event may trigger a process to log data in the data logging device 12.

Thus, in embodiments, the at least one sensor 14 is a first sensor to detect a first environmental event, and the device 12 further comprises a second sensor to detect a second environmental event. The or each sensor 14 of the data logging device 12 may sense/detect naturally-occurring external signals/events, and/or may sense artificial external signals such as electrical impulses, artificial light sources/illumination, chlorophyll, and electromagnetic fields/EM radiation, for example.

The data logging device 12 comprises a data store 16. In embodiments, the data store is a biological data store, which may comprise a nucleic acid strand. The nucleic acid strand may be a DNA (deoxyribonucleic acid) strand or an RNA (ribonucleic acid) strand. In embodiments, the biological data store may comprise a single strand of DNA or RNA, double-stranded (double helix) DNA or RNA, or triple-strand (triplex) DNA. DNA and RNA are naturally-occurring mechanisms to store information.

Artificial or engineered DNA (or RNA) may be used to store data in the sequence of the nucleic acid, and has a longevity and data density that is higher than current hard drive storage systems. Typically, custom-designed DNA molecules are used to store data, e.g. by employing an encoding scheme which maps digital data to sequences of nucleotides/bases, and the designed molecules are fabricated using DNA synthesis techniques. However, the typical technique may not be appropriate or readily performed within a data logging device.

Thus, in embodiments of the present techniques, the data store 16 may comprise a host chromosome (i.e. a DNA molecule) which is autonomously edited in situ within the data logging device 12 using biological techniques and thereby, used to log data. The host DNA molecule is edited in situ each time an external event is sensed by the or each sensor 14, by adding in a marker that indicates that the event has occurred. The same marker (i.e. a short sequence of nucleotides) is added for each event detected by a sensor 14. In embodiments where the data logging device 12 comprises multiple sensors 14, a different marker may be added to the host DNA molecule for each sensor. Thus, the host DNA molecule grows in length each time an event is detected by the or each sensor 14. The number of marker sequences which are added to the DNA molecule indicate the number of times a particular event was detected (e.g. a change in pH, salinity, chemical concentration, etc.)

In embodiments, the data store 16 may be any naturally-occurring biological or biochemical entity. For example, the data store 16 may comprise a naturally-occurring DNA molecule, e.g. a genomic DNA strand such as the M13mp18 virus DNA strand, or an RNA molecule. In embodiments, the data store 16 may comprise synthetic or engineered molecule or synthetic nucleic acid strand. In embodiments, the data store 16 may be an engineered or modified version of a naturally-occurring molecule.

The term “host chromosome” is used interchangeably herein with the terms “host DNA”, “host RNA”, “host”, “host nucleic acid”, “host nucleic acid strand” “host molecule”, and “host strand”, and is used generally to mean any naturally-occurring, synthetic, genetically-modified, or otherwise engineered nucleic acid.

The data logging device 12 comprises a write mechanism 18 to add the marker sequences into the host molecule upon detection of an event by sensor 14. The write mechanism 18 is configured to write into the host molecule at a single point/position in the host molecule, such that the writing takes place in an ordered, sequential manner. Specifically, the write mechanism 18 comprises means to nick/break the host molecule at the same location each time a write takes place, and the marker is inserted on the same side of the break during each write.

In embodiments, the write mechanism 18 comprises any suitable biological technique to identify a particular location on the host chromosome (e.g. a particular sequence of nucleotides), to nick/cut the host chromosome at this location, and to insert a marker in the host chromosome on one side of the nick. The write mechanism 18 may also comprise means to ligate (i.e. join) the fragmented host chromosome and marker together. For example, the write mechanism 18 may comprise a nicking enzyme (or nicking endonuclease) which may cut a DNA strand at a specific site (i.e. a restriction site), by detecting a particular sequence of nucleotides. The write mechanism 18 may comprise a ligase which helps to join together molecules, such as T4 DNA ligase which facilitates the joining of DNA strands.

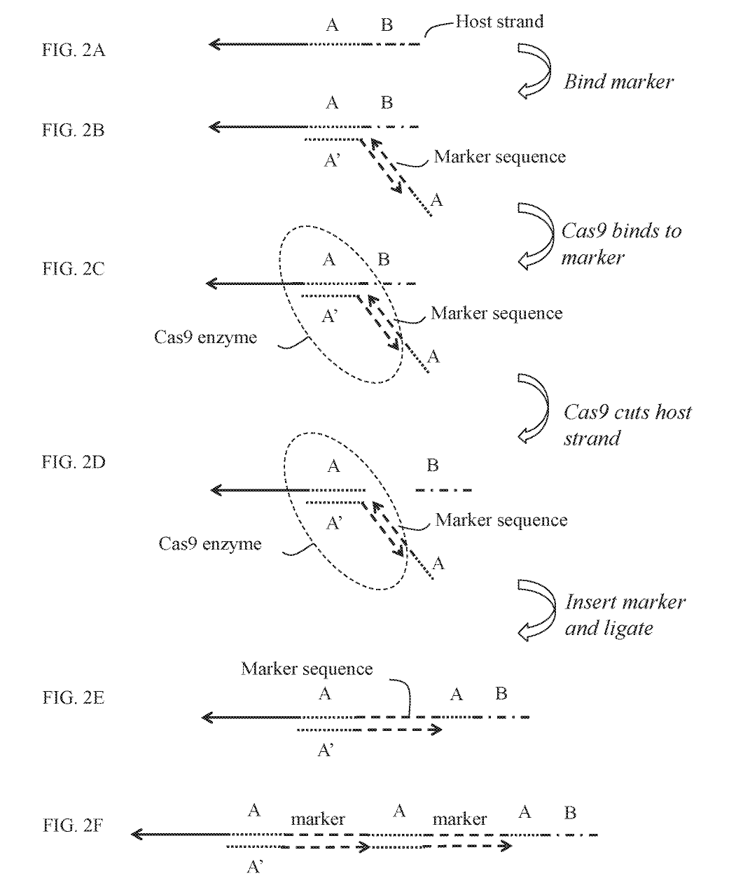

In embodiments, the write mechanism 18 may comprise a CRISPR/Cas9 system to write data (markers) into a nucleic acid strand. The CRISPR (clustered regularly interspaced short palindromic repeats)/Cas9 system is a genome editing tool which enables a DNA or RNA sequence to be edited by cutting out, replacing or adding nucleotides (or sequences of nucleotides) into the DNA or RNA sequence. In embodiments, the write mechanism 18 may comprise a CRISPR/Cas9 system. Cas9 is an enzyme which acts as molecular scissors and may be used to cut DNA or RNA strands at specific locations. The CRISPR/Cas9 system comprises a piece of RNA (known as guide RNA, or gRNA) which comprises a pre-designed sequence of nucleotides (e.g. around 20 bases long) and a scaffold sequence. The scaffold sequence is designed to bind to a specific complementary sequence in the DNA or RNA strand which is to be cut, and the pre-designed sequence guides the Cas9 enzyme to the position in the DNA/RNA strand that is to be cut. Following the cutting by Cas9, a marker may be written into/added to the nucleic acid strand on one side of the cut, and a mechanism is employed to join together the sequence fragments (e.g. the ligase enzyme). An advantage of using a write mechanism 18 that comprises a CRISPR/Cas9 system is that the guide DNA may be produced (or a concentration of the guide DNA increased) in response to the sensor 14 detecting an environmental event, such that the write process is triggered in response to an event being sensed. This process is described in more detail with respect to FIG. 2 below.

Thus, in embodiments, the data logging device 12 comprises at least one write mechanism 18 to, responsive to detection of the environmental event by sensor 14, write data into the biological data store 16.

The data logging device 12 further comprises at least one clocking mechanism 20 configured to add a timestamp to the biological data store 16 after each clock cycle of the clocking mechanism. The writing mechanism 18 described above inserts a marker (i.e. writes data) into a host chromosome each time an event has been sensed by sensor 14. The resultant data stored by the data logging device 12 is a count of how many times an event has been sensed by sensor 14. In embodiments, this count may provide useful information on its own. However, in embodiments, the count may be more useful if the time when each event occurs is also logged/recorded. For example, for a data logging device 12 which is deployed in an environment for several years, it may be useful to know if the events were sensed regularly or intermittently, or how many times the events take place on average within a certain period of time. Thus, adding in a timestamp or clock marker into the host chromosome in addition to the markers added in response to an event being sensed provides additional information about the frequency or regularity of event occurrence.

The clocking mechanism 20 may comprise a biological oscillator that has a particular (average) clock cycle. The clocking mechanism 20 may add a timestamp to the host chromosome once per clock cycle. The timestamp may be a specific pre-designed sequence of nucleotides which are added into the host chromosome in the same manner as a marker is added by the write mechanism 18. Thus, the host chromosome edited by the data logging device 12 comprises a series of timestamps and data markers. Depending on the average frequency and regularity of an event being sensed by sensor 14, there may be zero or more data markers between each pair of timestamps. The timestamps may be used to mark any period of time, from hours to days, to months or even years. The timestamps may be used to mark natural periods of time, such as a daylight cycle, day-night cycle, one or more seasonal cycles, etc. The required clock cycle period may be provided by selecting an appropriate biological oscillator.

It will be understood that a biological oscillator's clock cycle is an average clock cycle, or may be considered to have an average clock period. For example, a biological clock may periodically switch between states every 3 to 7 days, or every 24 hours. However, individual cycles may take place slightly faster or slightly slower. Thus, in embodiments, multiple data logging devices 12 may be deployed in an environment, which may multiply (e.g. via the cell division process if each data logging device 12 is natural or synthetic cell), such that probability density analysis may be performed to determine when events were recorded in the data store 16.

In embodiments, the data logging device 12 may comprise multiple clocking mechanisms, which may be used to mark different periods of time in the host chromosome (e.g. a coarse and a fine timestamp). Thus, the at least one clocking mechanism may comprise a first clocking mechanism operating at a first clock cycle, and a second clocking mechanism operating at a second clock cycle. The first clock cycle may be longer than the second clock cycle. In embodiments, the first and second clock cycles may be substantially the same length, such that the second clock is a back-up if the first clock fails, and/or provides a more reliable time recordal system. In embodiments, the first and second clock cycles may be identical or substantially identical but out of phase with each other. In embodiments, the first clocking mechanism and the second clocking mechanism provide a two-phase clock. In embodiments, the data logging device 12 may comprise multiple clocking mechanisms which together provide a multi-phase clock.

The system 10 of FIG. 1 further comprises a read mechanism 22, to read the data stored in the biological data store 16. In embodiments, the read mechanism 22 may comprise a sequencing device, e.g. a DNA sequencer. Once the stored sequence has been read, the sequence may be analyzed to locate the position of the timestamps and the markers, to determine how often an event was sensed, for example.

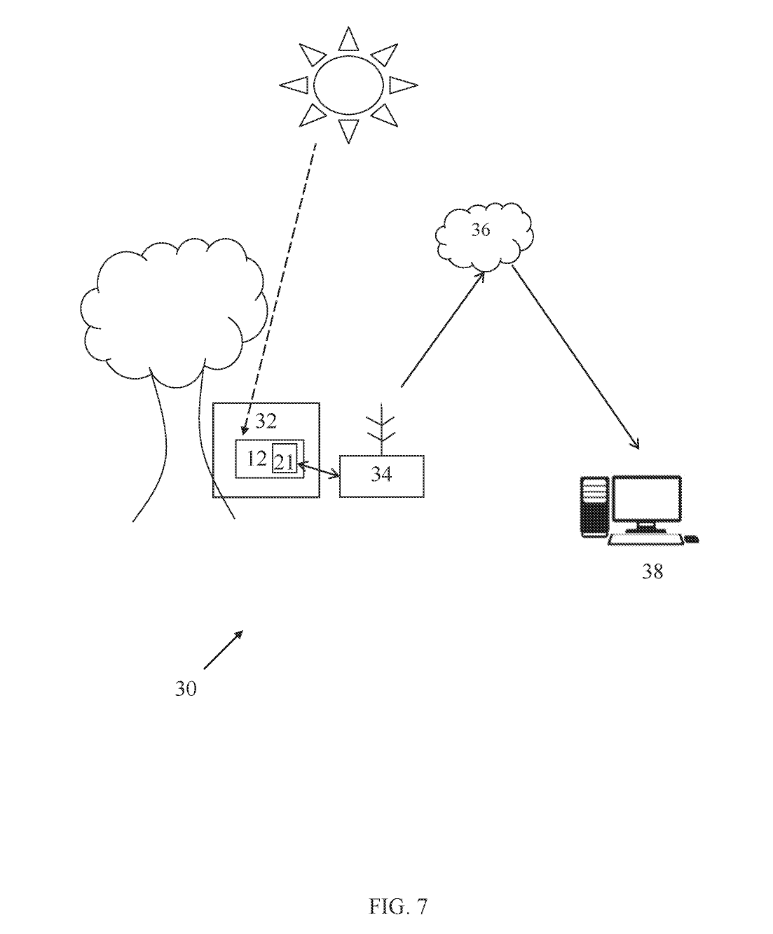

In embodiments, the data logging device may comprise a transmitter 21 to transmit a signal indicating a status of the device. The transmitter 21 may comprise one or more radio-labelled nucleotides or fluorescently-labelled nucleotides, for example, which may either be incorporated into the host chromosome during insertion of a marker or timestamp, or may be produced in response to external stimuli. The transmitter 21 may enable a status of the device to be communicated to a monitoring station, for example. For instance, if many writes have taken place, a fluorescent signal produced by fluorescently-labelled nucleotides in the marker may be high enough to be detected by an optical device located in the vicinity of the data logging device. The detection by the optical device may be communicated to a monitoring station to provide some feedback on the data logging device without needing to read the stored data. This is described in more detail below with reference to FIG. 7.

Thus, in the system 10, the read mechanism 22 may comprise a nucleic acid sequencing device, and/or an imaging device to receive a signal transmitted by the transmitter.

FIG. 2 illustrates example steps to log (i.e. write) data or to add a timestamp into a biological data store 16, using a CRISPR/Cas9 system as an example editing tool. FIG. 2a depicts a host nucleic acid strand (or a portion of a host chromosome). For the sake of simplicity, the host is depicted as a single strand of nucleic acid in FIG. 2, but it will be understood that the host may be a double-stranded nucleic acid. The host strand comprises a write block, or write position, which indicates where the host strand is to be cut and where a sensed data marker or a timestamp is to be inserted. The write block may comprise two portions, labelled A and B, which may each be around 10 bases/nucleotides long. In embodiments, the host strand is a single strand of nucleic acid, and in alternative embodiments, the host strand is double-stranded nucleic acid. FIG. 2 illustrates a single strand embodiment, which is merely exemplary and non-limiting.

In this illustrated embodiment, the marker or timestamp to be added into the host strand comprises a double-stranded complex having a sequence that identifies whether the complex is a marker complex or a timestamp complex. If the data logging device 12 comprises multiple sensors 14 and/or multiple clocking mechanisms 20, each is associated with an identifying sequence. The marker comprises a segment which is complementary to part of the write block sequence, and a segment which is identical to the same part of the write block sequence. As shown in FIG. 2b, in this example, the marker complex comprises a segment that is complementary to portion A of the write block (A′) and a segment that is the same as portion A. When the marker is added to the host strand, the A′ portion binds to portion A of the host strand, as depicted in FIG. 2b. The marker sequence functions as the guide for Cas9: the Cas9 enzyme binds to the marker sequence, and is thereby guided to the location in the host strand which is to be cut.

The Cas9 enzyme binds to the marker sequence, as depicted in FIG. 2c, and proceeds to cut the host strand in portion A, as depicted in FIG. 2d. The marker complex is now inserted into the host strand on one side of the cut site. A ligase or similar technique is employed to join together the fragments and form an edited host strand that contains the marker sequence, as depicted in FIG. 2e. Segment A of the marker complex is next to portion B of the host strand, such that the original write block sequence is formed again. This means that the next time a marker or timestamp is to be written into the host strand, it is inserted at the position of the write block. FIG. 2f depicts the host strand after two marker insertions, to illustrate how the write block is always reformed after each insertion, and how the series of markers grows in one direction along the strand.

The CRISPR/Cas9 system illustrated in FIG. 2 may be used for both the write mechanism and the clocking mechanism. A different Cas9 enzyme may be used for each mechanism (such that it binds to only one of the marker sequence or timestamp sequence).

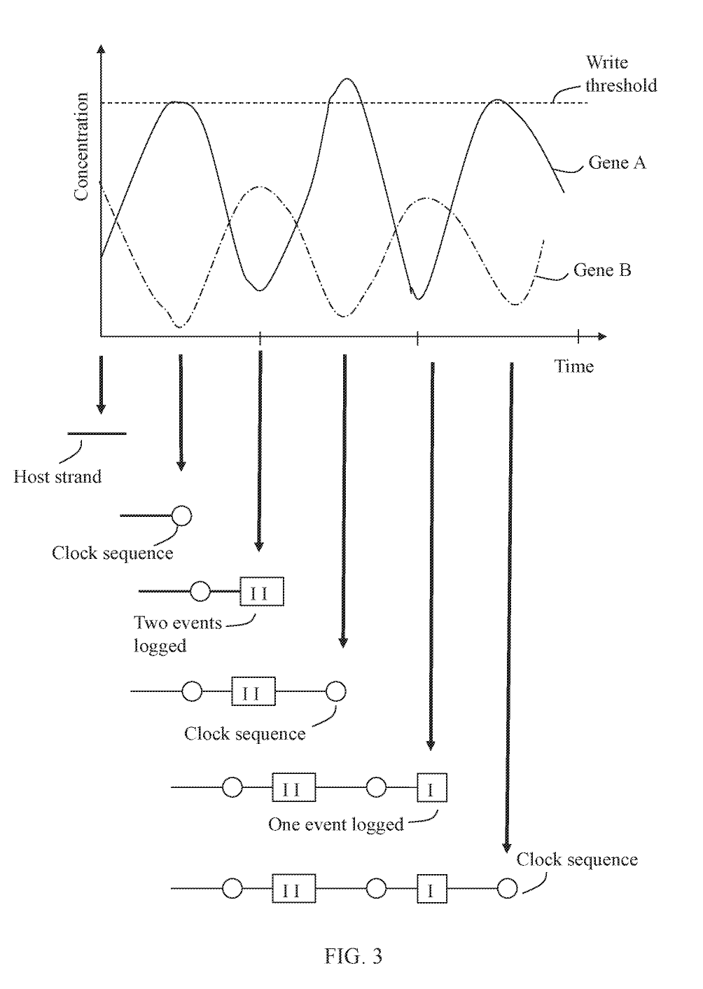

FIG. 3 illustrates an example clocking mechanism 20 comprising a two gene oscillator (graph), and a schematic of how data and timestamps are added to a biological data store 16. Here, the oscillator comprises a first gene (gene A) and a second gene (gene B). Both genes A and B are present within the data logging device 12 in some concentration. In the illustrated example, when the concentration of gene A reaches or exceeds a threshold concentration, the process to add a timestamp into the host chromosome is triggered. For example, when the concentration of gene A reaches the threshold value, the production of the timestamp complex is triggered. (For example, gene A may encode a transcription factor which up-regulates production of the timestamp complex). Once this has been produced, the timestamp complex binds to the host chromosome, and the Cas9 enzyme performs the task of cutting the host chromosome, as described above. Once the timestamp has been added to the host chromosome, the concentration of gene A decreases and the concentration of gene B increases. When the concentration of gene B reaches a particular threshold level, production of gene A is triggered. In this way, the changing concentrations of the two genes produces an oscillator, i.e. a clock.

Thus, in embodiments, the at least one clocking mechanism to add a timestamp to the biological data store after each clock cycle comprises a biological oscillator. In embodiments, the biological oscillator is a two-gene oscillator and comprises: a first gene encoding a first repressor protein; and a second gene encoding a second repressor protein; wherein the first repressor protein inhibits transcription of the second gene, and the second repressor protein inhibits transcription of the first gene.

FIG. 3 also shows a sketch of how a host strand may be edited to store data about sensed events and to store timestamps. A timestamp or clock sequence is added to the host chromosome at the write block position when the concentration of gene A reaches or exceeds a threshold value. Between timestamps, the sensor 14 of the data logging device 12 may detect two events. The write mechanism 18 adds two write/data markers into the host chromosome. Another clock sequence/timestamp is added when the concentration of gene A reaches the threshold value. This time, only one event is sensed by the sensor 14, and thus, only one write marker/data marker is added to the host chromosome. The final part of the sketch shows that a third timestamp is added to the host chromosome, and shows how time series data is logged into a biological data store. It will be understood that this sketch is merely illustrative. In some cases, no events may be sensed by the sensor between time stamps.

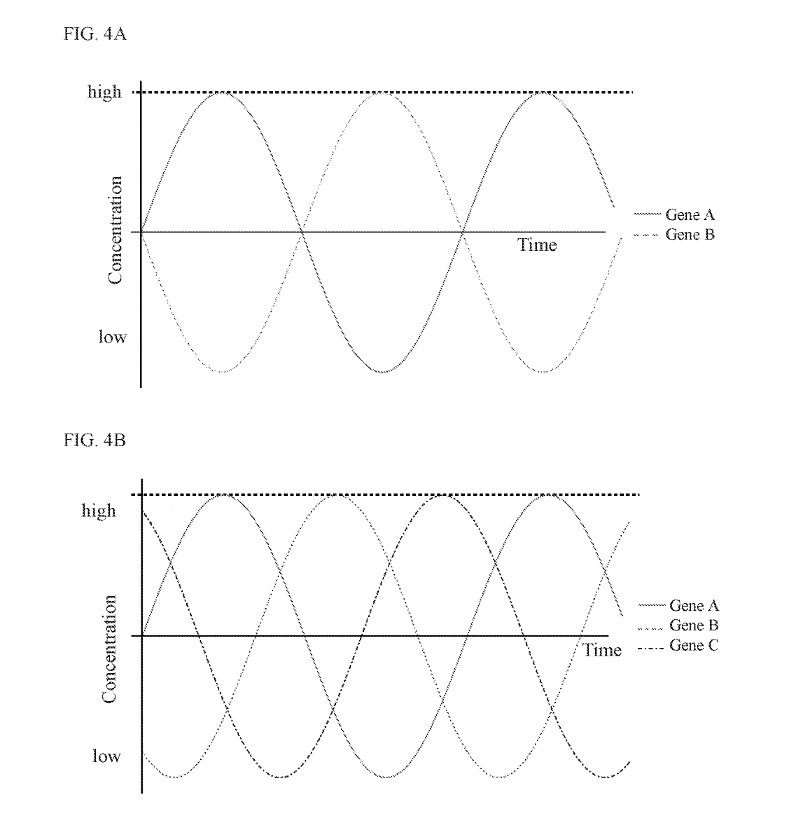

FIG. 4a illustrates another example clocking mechanism comprising a two gene oscillator. Compared to the example depicted in FIG. 3, here, the gene concentrations may fluctuate periodically between the same high and low concentrations. (In FIG. 3, the high concentration level of gene B was lower than that of gene A). There are many other possible configurations of a two-gene oscillator.

FIG. 4b illustrates an example clocking mechanism comprising a three gene oscillator. Here, the oscillator comprises a first gene (gene A), a second gene (gene B), and a third gene (gene C). All three genes A to C may periodically fluctuate between high and low concentrations. Although FIG. 4b shows that all three genes reach the same high and low concentration levels, it will be understood that this is merely exemplary and in some cases the high and low concentrations for each gene may be different.

All three genes A, B and C are present within the data logging device 12 in some concentration, and the cross-regulating between the three genes may form an oscillating, non-damped system.

In an example three-gene oscillator, gene A may comprise a binding site for transcription factor A, which promotes transcription of gene A. Gene A may comprise a section of nucleotides that represent the timestamp for the clocking mechanism. When the concentration of gene reaches or exceeds a threshold concentration (which may be a high or a low concentration level), the timestamp nucleotides are transcribed into RNA along with the rest of gene A, and are then inserted into the host strand via the process described above. Therefore, gene A also comprises the portion of the write block sequence which is required to re-establish the write block after insertion (ready for the next write into the host strand).

Gene A may comprise a transcription factor for gene B, which upregulates the production of gene B. Gene A may comprise a repressor protein capable of down-regulating the transcription factor for gene C. This protein may be an operator protein, such as the lac operon, or may be a ubiquitin protein that targets transcription factor proteins for gene C for fast degradation. Thus, when gene A is transcribed and reaches a particular concentration, it triggers gene C concentration to decrease and gene B concentration to increase.

Gene B may comprise a binding site for transcription factor B, which promotes transcription of gene B. Gene B may comprise a transcription factor for gene C, which upregulates the production of gene C. Gene B may comprise a repressor protein capable of down-regulating the transcription factor for gene A. This protein may be an operator protein, such as the lac operon, or may be a ubiquitin protein that targets transcription factor proteins for gene A for fast degradation. Thus, when gene B is transcribed and reaches a particular concentration, it triggers gene C concentration to increase and gene A concentration to decrease.

Gene C may comprise a binding site for transcription factor C, which promotes transcription of gene C. Gene C may comprise a transcription factor for gene A, which upregulates the production of gene A. Gene C may comprise a repressor protein capable of down-regulating the transcription factor for gene B. This protein may be an operator protein, such as the lac operon, or may be a ubiquitin protein that targets transcription factor proteins for gene B for fast degradation. Thus, when gene C is transcribed and reaches a particular concentration, it triggers gene A concentration to increase and gene B concentration to decrease.

The oscillations are shown in FIG. 4b. When the concentration of gene A reaches or exceeds a threshold concentration, the process to add a timestamp into the host chromosome is triggered. In embodiments, the whole of gene A is added into the host chromosome. In other embodiments, only the timestamp and write block portion are added to the host chromosome. Once the timestamp has been added to the host chromosome, the concentration of gene A decreases, the concentration of gene B increases and the concentration of gene C decreases. When the concentration of gene B reaches a particular threshold level, production of gene C is triggered. When the concentration of gene C reaches a particular threshold level, production of gene A is triggered. In this way, the changing concentrations of the three genes produces an oscillator, i.e. a clock.

Thus, in embodiments, the biological oscillator comprises: a first gene encoding a first repressor protein and a first transcription factor; a second gene encoding a second repressor protein and a second transcription factor; and a third gene encoding a third repressor protein and a third transcription factor; wherein: the first repressor protein inhibits transcription of the third gene, the first transcription factor up-regulates production of the second gene, the second repressor protein inhibits transcription of the first gene, the second transcription factor up-regulates production of the third gene, the third repressor protein inhibits transcription of the second gene, and the third transcription factor up-regulates production of the first gene. A three gene oscillator may be less likely to become a damped system than a two gene oscillator, and therefore may be better suited as a reference clock/clocking mechanism in the data logging device 12, particularly if the device 12 is to be deployed for long periods of time.

In either the two-gene or three-gene oscillator (or any other oscillator), the first gene may comprise the timestamp (or a transcription factor to produce the timestamp complex), wherein the timestamp is added to the biological data store per clock cycle when a concentration of the first gene reaches a threshold value.

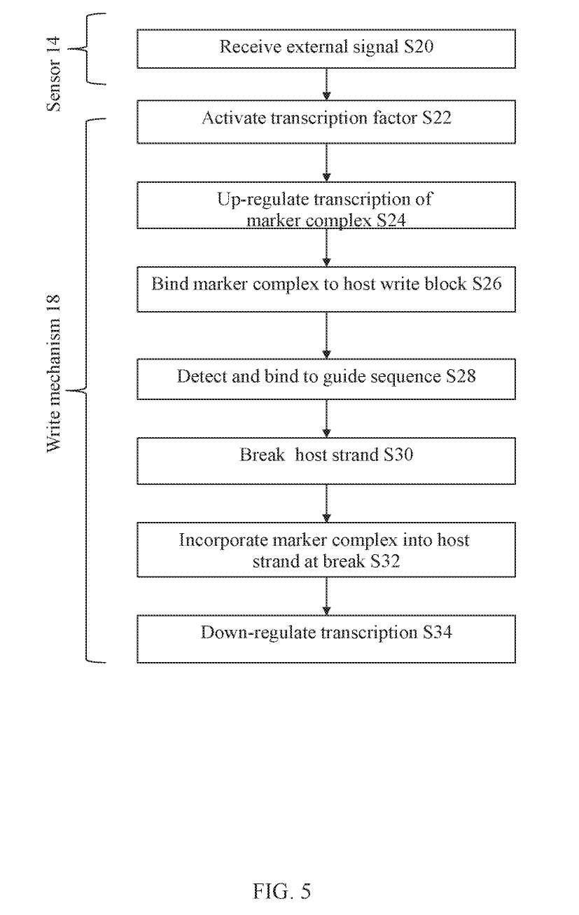

FIG. 5 is a flow diagram of example steps performed by the data logging device 12 to write data in response to sensing an event. Sensor 14 receives an external signal which is indicative of an environmental event taking place (step S20). For example, the external signal may be a particular wavelength of electromagnetic radiation, or a decrease in pH. Receiving this signal triggers the process to write data into host chromosome.

At step S22, a transcription factor associated with the marker (data) complex is activated. Activation of the transcription factor causes up-regulation of the marker complex (i.e. the production of the marker complex begins) (step S24). When the marker complex has been transcribed or reaches a particular concentration, it binds at least partially to the write block in the host chromosome (step S26), as described earlier with reference to FIG. 2.

The Cas9 enzyme of the write mechanism 18 detects the guide portion of the marker complex, and binds to the guide portion/sequence (step S28). In this way, the Cas9 enzyme is brought into close proximity with the write block of the host chromosome such that it can break or cut the host strand (step S30). The write mechanism 18 incorporates the marker complex into the host strand at the cut site (step S32). Insertion and ligation triggers the down-regulation of the transcription of the marker complex (step S34) as production of the marker complex is no longer required until the next write.

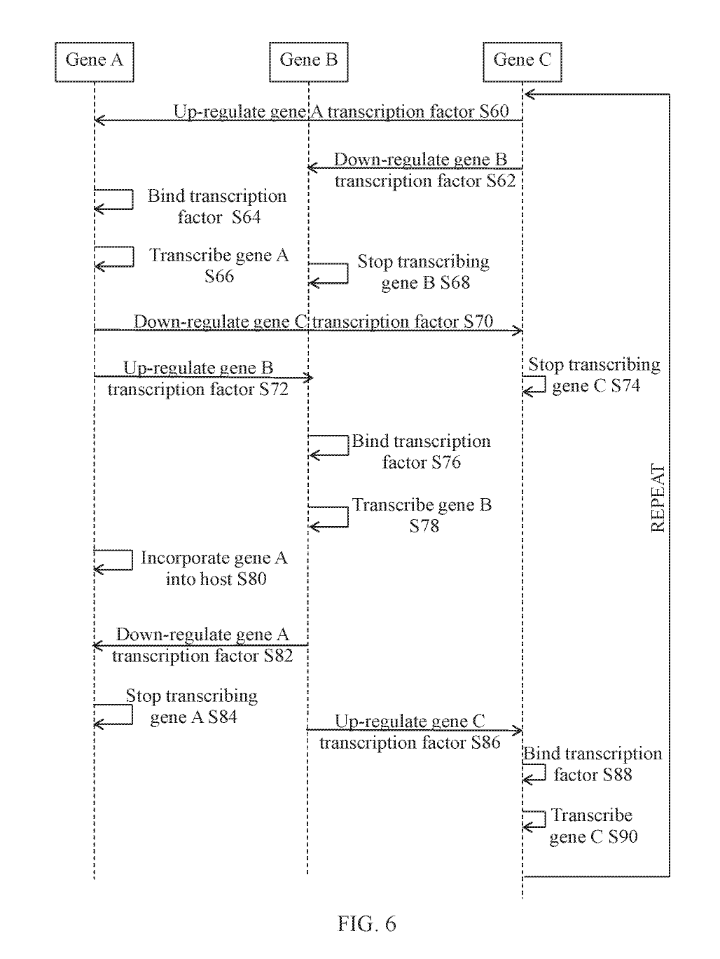

FIG. 6 is a schematic diagram of how the example three gene oscillator described above with respect to FIG. 4b may be used to add timestamps to a biological data store. At step S60, gene C has reached a threshold concentration (which may be a high or low concentration), and transcription factor for gene A (TF_A) is produced. This results in the production or up-regulation of gene A. Substantially simultaneously, gene C encodes for a repressor protein that down regulations the transcription factor for gene B, such that gene B is down-regulated (step S62). In response to the up-regulation of TF_A, TF_A binds to the binding site on gene A (step S64) which results in gene A being transcribed (step S66). In response to the down-regulation of TF_B, gene B stops being transcribed (step S68). The production of gene A and non-production of gene B may be triggered substantially simultaneously.

After some time, gene A reaches a threshold concentration (which may be a high or low concentration). This causes the transcription factor for gene B (TF_B) to be produced, which results in the production or up-regulation of gene B (step S72). Substantially simultaneously, gene A encodes for a repressor protein that down regulations the transcription factor for gene C (TF_C), such that gene C is down-regulated (step S70). When gene A reaches the threshold concentration, the mechanism to insert gene A (and/or the timestamp it encodes) into the host chromosome is triggered, such that gene A is incorporated into the host (step S80).

In response to the up-regulation of TF_B, TF_B binds to the binding site on gene B (step S76) which results in gene B being transcribed (step S78). In response to the down-regulation of TF_C, gene C stops being transcribed (step S74). The production of gene B and non-production of gene C may be triggered substantially simultaneously. This may take place at the same time that gene A is being incorporated into the host.

After some time, gene B reaches a threshold concentration (which may be a high or low concentration). This causes the transcription factor for gene C (TF_C) to be produced, which results in the production or up-regulation of gene C (step S86). Substantially simultaneously, gene B encodes for a repressor protein that down regulations the transcription factor for gene A (TF_A), such that gene A is down-regulated (step S82). In response to the up-regulation of TF_C, TF_C binds to the binding site on gene C (step S88) which results in gene C being transcribed (step S90). In response to the down-regulation of TF_A, gene A stops being transcribed (step S84). The production of gene C and non-production of gene A may be triggered substantially simultaneously.

After some time, gene C reaches a threshold concentration, and the cycle returns to step S60. In this way, the changing concentrations of the three genes produces an oscillator, i.e. a clock.

The data logging device 12 described herein may be used for a wide variety of purposes. For example, the data logging device 12 may be used to sense particular events on land and in bodies of water, and/or be appended to (or dispensed from) aircraft to sense events in the air (e.g. concentrations of airborne particulates). In one example, the data logging device 12 may be used to monitor daylight. In this case, sensor 14 may be a light sensitive receptor. When the light sensitive receptor detects a particular wavelength of light, or a particular intensity of light, it causes the production of a marker complex, as explained above with reference to FIG. 5. In another example, the data logging device 12 may be used to monitor pH levels, or changes in pH. In this example, the sensor 14 may be a proton-sensitive protein. In another example, the data logging device 12 may be appended to animals in the wild, e.g. as part of a tracking device or tag, and the sensor 14 may detect changes in particular hormones.

Further example uses of the data logging device 12 described herein include: measuring ocean temperature for the purpose of predicting the on-set and duration of El Niño; monitoring ice-sheet thickness by sensing light which has penetrated through ice—the data logging device 12 may need to be placed below an ice-sheet to do so; measuring characteristics of soil, such as pH or mineral concentration, particularly in an agricultural setting; and effluent monitoring in cities.

In embodiments, multiple data logging devices 12 may be placed into a suitable receptacle and placed in an environment to be monitored. For example, a receptacle containing multiple data logging devices 12 may be inserted into soil in a field to measure characteristics of the soil, or may be attached to a tree to measure light and/or concentrations of air-borne particles or ions, or may be appended to an airplane or ship and used to monitor airborne particles or salinity, etc. in embodiments, the devices 12 may be applied to clothing (e.g. by being impregnated into the fibres of the clothing), and used to monitor characteristics of the wearer of the clothing. Placing the data logging devices 12 into a receptacle may simplify the process to locate the devices 12 at a later stage for analysis, and may ensure the devices 12 remain in the environment they are being used to monitor.

Additionally or alternatively, the data logging devices 12 may be dispersed into an environment. For example, if the devices 12 are being used to monitor soil characteristics, the devices 12 may be dispersed across a field using a ‘crop duster’ type mechanism. If the devices 12 are being used to monitor pH or salinity or pollution levels in the ocean, the devices 12 may be dispersed into an ocean. In these examples, the devices 12 may be dispersed over a large area, and here, the self-replication process is important for recovering the devices later for analysis. For example, if the devices 12 have replicated enough, a sample of ocean water may contain at least trace amounts of the devices 12. Processes to amplify the devices 12 and/or the biological data store 16 contained therein, may be used to increase the concentration to a sufficient level for analysis.

In each example, the sensor 14 is selected according to the event that is to be sensed/monitored. Many features of the data logging device 12 remain the same across different uses of the device 12, such as the host chromosome, the marker complex and the timestamp complex. In embodiments, it may be useful to use different marker sequences for each sensor type, so that when the stored data in the biological data store is read and analyzed, it can be readily determined what the sensor was sensing. Alternatively, a different host chromosome may be used for each sensor type.

FIG. 7 is a schematic diagram of an Internet of Things system comprising the data logging device 12 of the present techniques. The system shown in FIG. 7 is merely exemplary. The system 30 comprises a receptacle 32 which houses multiple data logging devices 12. (Only one device 12 is shown in the FIG. for the sake of simplicity). Each data logging device 12 comprises a transmitter 21 (as well as the other elements shown in FIG. 1). The data logging devices 12 have been deployed in an environment. In this example, the receptacle 32 containing the data logging devices has been placed on or in the vicinity of a tree, and is being used to monitor daylight cycles or sunlight intensity/levels. The transmitter 21 may take the form of a nucleotide which emits light or fluoresces when stimulated by an external signal (e.g. light of a particular wavelength). The transmitter 21 may be contained within each marker that is added to the data store 16 in the device 12. Accordingly, the intensity of the light emitted by the transmitter 21 upon stimulation by an external signal may provide some information about how many events have been sensed by the device 12.

The system 30 comprises equipment 34 which is configured to apply a signal to the devices 12 and receive a signal from the transmitters 21. The equipment 34 may comprise an optical source and detector, and means to communicate with a server 38 that is located remote to the devices 12. The equipment 34 may be configured to send the received data/signal to a monitoring station or server 38, via a communication network 36 (e.g. via a mobile network). Accordingly, data on the number of events that have been recorded may be obtainable and analyzable without needing to retrieve the devices 12 and/or without needing to read/sequence the biological data store 16.

In embodiments, the transmitter 21 may be contained within each timestamp that is added to the data store 16 in the device 12. Accordingly, the duration of operation of the devices 12 may be obtainable.

In embodiments, the server 38 may obtain data on the status of each receptacle 32 deployed in a particular environment, such that near real-time data may be obtainable across an environment without needing to be in the environment or needing to sequence the data stores 16 of each device 12 in every receptacle 32. This may enable on-going data collection, as well as long-term data logging.

Further embodiments are set out in the following numbered clauses:

1. A data logging device comprising: at least one sensor to detect an environmental event; a biological data store; and at least one write mechanism to, responsive to detection of the environmental event, write data into the biological data store.

2. The data logging device as recited in clause 1 further comprising: at least one clocking mechanism to add a timestamp to the biological data store after each clock cycle.

3. The data logging device as recited in clause 2 wherein the clocking mechanism comprises a biological oscillator.

4. The data logging device as recited in clause 3 wherein the biological oscillator comprises: a first gene encoding a first repressor protein; and a second gene encoding a second repressor protein; wherein the first repressor protein inhibits transcription of the second gene, and the second repressor protein inhibits transcription of the first gene.

5. The data logging device as recited in clause 3 wherein the biological oscillator comprises: a first gene encoding a first repressor protein and a first transcription factor; a second gene encoding a second repressor protein and a second transcription factor; and a third gene encoding a third repressor protein and a third transcription factor; wherein: the first repressor protein inhibits transcription of the third gene, the first transcription factor up-regulates production of the second gene, the second repressor protein inhibits transcription of the first gene, the second transcription factor up-regulates production of the third gene, the third repressor protein inhibits transcription of the second gene, and the third transcription factor up-regulates production of the first gene.

6. The data logging device as recited in clause 3 wherein the biological oscillator comprises a multiple of three genes.

7. The data logging device as recited in clause 4, 5 or 6 wherein the first gene comprises the timestamp, wherein the timestamp is added to the biological data store per clock cycle when a concentration of the first gene reaches a threshold value.

8. The data logging device as recited in any preceding clause wherein the biological data store comprises at least one nucleic acid strand.

9. The data logging device as recited in clause 8 wherein the nucleic acid strand comprises a DNA strand.

10. The data logging device as recited in clause 8 wherein the nucleic acid strand is an RNA strand.

11. The data logging device as recited in clause 8 wherein the nucleic acid strand is synthetic.

12. The data logging device as recited in any one of clauses 8 to 11 wherein the write mechanism comprises a CRISPR/Cas9 system to write data into the nucleic acid strand.

13. The data logging device as recited in any one of clauses 8 to 11, when dependent on clause 7, wherein the clocking mechanism comprises a CRISPR/Cas9 system to add the timestamp to the nucleic acid strand.

14. The data logging device as recited in any one of clauses 8 to 13 wherein the nucleic acid strand comprises a write block which defines where the CRISPR/Cas9 system cuts the nucleic acid strand.

15. The data logging device as recited in any preceding clause wherein the sensor to detect an environmental event comprises a receptor molecule.

16. The data logging device as recited in any preceding clause wherein the sensor to detect an environmental event comprises a cell surface receptor.

17. The data logging device as recited in clause 15 or 16 wherein the receptor detects changes in any one of: pH, light, wavelength, electromagnetic radiation, presence of an element, concentration of an element, concentration of an ion, and concentration of a molecule.

18. The data logging device as recited in any preceding clause wherein the at least one sensor comprises a first sensor to detect a first environmental event, and a second sensor to detect a second environmental event.

19. The data logging device as recited in any one of clauses 2 to 18 wherein the at least one clocking mechanism comprises a first clocking mechanism operating at a first clock cycle, and a second clocking mechanism operating at a second clock cycle.

20. The data logging device as recited in clause 19 wherein the first clock cycle is longer than the second clock cycle.

21. The data logging device as recited in clause 19 wherein the first clocking mechanism and the second clocking mechanism form a multi-phase clock.

22. The data logging device as recited in any preceding clause further comprising a transmitter to transmit a signal indicating a status of the device.

23. The data logging device as recited in clause 22 wherein the transmitter comprises one or more radio-labelled or fluorescently-labelled nucleotides.

24. The data logging device as recited in any preceding clause wherein the device is one of: a synthetic cell, a natural cell, and an engineered cell.

25. The data logging device as recited in any preceding clause wherein the device is autonomous.

26. The data logging device as recited in any preceding clause wherein the device is one or more of: self-assembling, self-maintaining, and self-replicating.

27. A system comprising: a data logging device according to any one of clauses 1 to 26; and at least one read mechanism to read data stored in the biological data store.

28. The system as recited in clause 27 wherein the read mechanism comprises a nucleic acid sequencing device.

29. The system as recited in clause 27 or 28 wherein the read mechanism comprises a device to receive a signal transmitted by the transmitter.

30. A method of logging data, comprising: detecting, using at least one sensor, an environmental event; writing, responsive to the detecting, data into a biological data store.

31. The method as claimed in claim 30 further comprising: adding, using a clocking mechanism, a timestamp to the biological data store after each clock cycle of the clocking mechanism.

Those skilled in the art will appreciate that while the foregoing has described what is considered to be the best mode and where appropriate other modes of performing present techniques, the present techniques should not be limited to the specific configurations and methods disclosed in this description of the preferred embodiment. Those skilled in the art will recognize that present techniques have a broad range of applications, and that the embodiments may take a wide range of modifications without departing from the any inventive concept as defined in the appended claims.

1. A data logging device comprising:
at least one sensor to detect an environmental event;
a biological data store; and
at least one write mechanism to, responsive to detection of the environmental event, write data into the biological data store.

2. The data logging device as claimed in claim 1 further comprising:
at least one clocking mechanism to add a timestamp to the biological data store after each clock cycle.

3. The data logging device as claimed in claim 2 wherein the clocking mechanism comprises a biological oscillator.

4. The data logging device as claimed in claim 3 wherein the biological oscillator comprises:
a first gene encoding a first repressor protein; and
a second gene encoding a second repressor protein;
wherein the first repressor protein inhibits transcription of the second gene, and the second repressor protein inhibits transcription of the first gene.

5. The data logging device as claimed in claim 4 wherein the first gene comprises the timestamp, wherein the timestamp is added to the biological data store per clock cycle when a concentration of the first gene reaches a threshold value.

6. The data logging device as claimed in claim 3 wherein the biological oscillator comprises:
a first gene encoding a first repressor protein and a first transcription factor;
a second gene encoding a second repressor protein and a second transcription factor; and
a third gene encoding a third repressor protein and a third transcription factor;
wherein:
the first repressor protein inhibits transcription of the third gene,
the first transcription factor up-regulates production of the second gene,
the second repressor protein inhibits transcription of the first gene,
the second transcription factor up-regulates production of the third gene,
the third repressor protein inhibits transcription of the second gene, and
the third transcription factor up-regulates production of the first gene.

7. The data logging device as claimed in claim 3 wherein the biological oscillator comprises a multiple of three genes.

8. The data logging device as claimed in claim 6 wherein the first gene comprises the timestamp, wherein the timestamp is added to the biological data store per clock cycle when a concentration of the first gene reaches a threshold value.

9. The data logging device as claimed in claim 1 wherein the biological data store comprises a nucleic acid strand.

10. The data logging device as claimed in claim 9 wherein the nucleic acid strand comprises a DNA strand.

11. The data logging device as claimed in claim 9 wherein the nucleic acid strand comprises an RNA strand.

12. The data logging device as claimed in claim 9 wherein the nucleic acid strand comprises synthetic.

13. The data logging device as claimed in claim 9 wherein the write mechanism comprises a CRISPR/Cas9 system to write data into the nucleic acid strand.

14. The data logging device as claimed in claim 9 wherein the data logging device comprises at least one clocking mechanism to add a timestamp to the biological data store after each clock cycle, the clocking mechanism comprising a CRISPR/Cas9 system to add the timestamp to the nucleic acid strand.

15. The data logging device as claimed in claim 9 wherein the nucleic acid strand comprises a write block which defines where a CRISPR/Cas9 system cuts the nucleic acid strand.

16. The data logging device as claimed in claim 1 wherein the sensor to detect an environmental event comprises a receptor molecule.

17. The data logging device as claimed in claim 1 wherein the sensor to detect an environmental event comprises a cell surface receptor.

18. The data logging device as claimed in claim 13 wherein the cell surface receptor detects changes in any one of: pH, light, wavelength, electromagnetic radiation, presence of an element, concentration of an element, concentration of an ion, and concentration of a molecule.

19. The data logging device as claimed in claim 1 wherein the at least one sensor comprises a first sensor to detect a first environmental event, and the device further comprises a second sensor to detect a second environmental event.

20. The data logging device as claimed in claim 2 wherein the at least one clocking mechanism comprises a first clocking mechanism operating at a first clock cycle, and the device further comprises a second clocking mechanism operating at a second clock cycle.

21. The data logging device as claimed in claim 20 wherein the first clock cycle is longer than the second clock cycle.

22. The data logging device as claimed in claim 20 wherein the first clocking mechanism and the second clocking mechanism form a multi-phase clock.

23. The data logging device as claimed in claim 1 further comprising a transmitter to transmit a signal indicating a status of the device.

24. The data logging device as claimed in claim 23 wherein the transmitter comprises one or more radiolabelled or fluorescently-labelled nucleotides.

25. The data logging device as claimed in claim 1 wherein the device is one of: a synthetic cell, a natural cell, and an engineered cell.

26. The data logging device as claimed in claim 1 wherein the device is autonomous.

27. The data logging device as claimed in claim 1 wherein the device is one or more of: self-assembling, self-maintaining, and self-replicating.

28. A system comprising:
a data logging device comprising:
at least one sensor to detect an environmental event;
a biological data store; and
at least one write mechanism to, responsive to detection of the environmental event, write data into the biological data store; and
at least one read mechanism to read data stored in the biological data store.

29. The system as claimed in claim 28 wherein the read mechanism comprises a nucleic acid sequencing device.

30. The system as claimed in claim 29 wherein the read mechanism comprises a device to receive a signal transmitted by the transmitter.

31. A method of logging data, comprising:
detecting, using at least one sensor, an environmental event;
writing, responsive to the detecting, data into a biological data store.

32. The method as claimed in claim 31 further comprising:
adding, using a clocking mechanism, a timestamp to the biological data store after each clock cycle of the clocking mechanism.
